(19) United States
(12) Patent Application Publication
(10) Pub. No.: US 2005/0276869 A1
(43) Pub. Date: Dec. 15, 2005

(54) APPETITE-SUPPRESSING, LIPASE-INHIBITING HERBAL COMPOSITION

(75) Inventor: James S. Bronner, Atlanta, GA (US)

Correspondence Address: RICHARD D. FUERLE, 1711 W. RIVER RD., GRAND ISLAND, NY, 14072, US

(73) Assignee: CENTURY SYSTEMS, Atlanta, GA (US)

(21) Appl. No.: 10/710,022

(22) Filed: Jun. 14, 2004

(51) Int. Cl. A61K 35/78

(52) U.S. Cl. 424/725; 424/757

(57) ABSTRACT

An herbal composition for suppressing appetite and inhibiting the formation of lipases contains water, about 30 to about 1000 mg of Hoodia gordonii per ¼ ounce of water, and about 30 to about 1000 mg of Cassia nomame per ¼ ounce of water. A serving of about 5 to about 15 cc of water may contain about 100 to about 300 mg of Hoodia gordonii, about 100 to about 300 mg of Cassia nomame, a sufficient amount of an acid, such as citric acid, to lower the pH below about 4.5, about 50 to about 100 mg of a flavoring agent, about 1800 to about 2600 mg of a flavor enhancer, and about 0.004 to about 0.12 mg of menthol. A convenient sealable container for carrying the composition holds about 8 to about 16 ounces. To lose weight, at least one serving of the composition is drunk twice a day before eating.

### BACKGROUND OF INVENTION

This invention relates to an herbal composition for suppressing appetite and for inhibiting the formation of lipases in the human body. In particular, it relates to an aqueous composition of the herbs Hoodia gordonii and Casia nomame.

The excessive accumulation of body fat (i.e. obesity) can be dangerous as it has been linked to health problems such as Type II diabetes, hypertension, hyperlipidemia, coronary heart disease, stroke, breast and colon cancer, sleep apnea, gallbladder disease, gastroesophogeal reflux disease, fatty liver, gout, and thromboembolism. Despite increased awareness among Americans that obesity increases these health risks, the prevalence of obesity in the United States has more than doubled since the turn of the century. (The Merck Manual, 15th ed. page 952.) Unfortunately, losing weight and keeping it off are very difficult for most individuals. Weight gain occurs when an individual's caloric intake exceeds the number of calories he expends as energy. Therefore, to lose weight, a person must consume fewer calories than he burns as energy. While many programs exist to help meet this goal, an increasing number of Americans are failing to do so.

Currently, 64.5 percent of U.S. adults, age 20 years and older, are overweight and 30.5 percent are obese. Severe obesity prevalence is now 4.7 percent, up from 2.9 percent reported in the 1988-1994. (National Health and Nutrition Examination Survey (NHANES) by the Centers for Disease Control and Prevention (CDC)) Thus, there is a need for an easy and effective method to promote weight loss.

### SUMMARY OF INVENTION

This invention helps people to lose weight through natural appetite suppression and natural blockage of the absorption of fat, while avoiding potentially negative side effects that are associated with certain highly stimulative dependent supplements. I have found that an aqueous composition of two herbs, Hoodia gordonii and Casia nomame, is effective in suppressing appetite and inhibiting the formation of lipases in the human body. Surprisingly, the composition of this invention is more effective than the same amount of those herbs taken in capsule form. While I do not wish to be bound by any theories, I believe that if the composition of this invention is taken immediately before a meal, it enters the bloodstream faster than it would from a capsule because of the increased time required for the digestive system to break down the gelatin capsule and dissolve the active ingredients. Also, the herbs may be slowly released from the capsules or may be released lower in the intestinal tract, where they are not absorbed as effectively. Absorption time is a crucial factor in the function of this composition because the actions of these two herbs are appetite suppressing and lipase inhibiting, which affect the amount of food eaten and digested. If most of the meal is consumed before the active ingredients enter the bloodstream, then it is too late to suppress appetite.

Another advantage of the composition of this invention over the same herbs in capsule form is that it is easy to remember to take the composition immediately before eating but more difficult to remember to take a capsule about twenty minutes before eating, to allow time for the capsule to dissolve. Moreover, the time required to dissolve the capsule may vary with different individuals.

I have further found that a particular variety of those herbs is more effective than other varieties.

### DETAILED DESCRIPTION

The herbal composition of this invention is a mixture of the herbs Hoodia gordonii and Casia nomame in water. The amount of water per serving (a dose) may vary as desired, but about 5 to about 15 cc is preferred. For the purpose of calculating amounts, ¼ ounce is used as a serving amount. Various optional components may also be present in the composition.

Any portion of the herbs Hoodia gordonii and Casia nomame may be used, including the leaves, stems, flowers, and roots. Not all sources of Hoodia gordonii are equally effective. I have found that Hoodia gordonii that has a peak at 1600 wave numbers of at least 0.05 absorbance units in a Fourier Transform Near Infrared Spectrometer (FTNIS) is especially effective. That variety is commercially available from Ashley Industries in Round Rock, Tex. The herbs are prepared by drying and fine milling so that they have the consistency of a powder. The amount of each herb in each serving may be from about 30 to about 1000 mg. The use of more herb is unnecessary and of little additional benefit and less herb may not be sufficiently effective; preferably, the amount of each herb is about 100 to about 300 mg per serving.

In addition to the two herbs, certain optional components may be added to the composition. It is preferably to include an acid in order to lower the pH to below about 4.5, which deters bacterial action that may degrade the composition. A pH between about 3.0 and about 4.5 is preferred as a lower pH is unnecessary and may be too acidic to drink and a higher pH may be less effective. Acids useful in lowering the pH include citric acid, ascorbic acid, acetylsalicylic acid (aspirin), hydrochloric acid, acetic acid, lactic acid, and malic acid. Citric acid is preferred as it has a pleasant taste and is accepted by the public. About 23 to about 35 mg of citric acid per ¼ ounce may be used. Alternatively, a preservative may be used instead of lowering the pH.

The composition may also include a flavoring agent to give it a pleasant taste. Examples of suitable flavoring include cherry, grape, orange, lemon, and strawberry; natural cherry flavor is preferred. The amount of flavoring agent depends upon the strength of the flavor, but may be about 50 to about 100 mg per serving.

A flavor enhancer may also be included in the composition to improve its flavor and texture. Suitable flavor enhancers include glycerin, stevia extract, and crystalline fructose. Glycerin is preferred because it produces a better tasting composition. The amount of flavor enhancer may be about 1800 to about 2600 mg per serving. Less flavor enhancer may not be effective and more may be unnecessary.

The composition may also include about 0.004 to about 0.12 mg of menthol per serving to inhibit microbial action and improve the flavor.

To prepare the composition, the dried herbs are comminuted and milled and are mixed with water and the optional components. To use the composition, it is drunk before a meal, preferably twice a day immediately before eating. If it is drunk earlier, its effect may have worn off and the person taking it may consume more food than he otherwise would have and, if it is taken after the meal has begun, it may not be absorbed in time to suppress the person's appetite. For an adult weighing up to 180 lbs the recommended dosage is 300 mg of Hoodia gordonii and 400 mg of Cassia nomame per day, i.e., two servings per day. For an adult weighting more than 180 lbs the recommended dosage is 600 mg Hoodia gordonii and 800 mg Cassia nomame per day, i.e., four servings per day. Since these herbs are believed to be non-toxic, there are no established maximum amounts. The herbs are not recommended for children, although no harmful effects on children have been observed.

The composition is preferably packaged in a small, easy-to-carry sealable container so that a person can carry it in a pocket or purse, drinking some just before eating. A container size holding about 8 to about 16 ounces is preferred.

The following examples further illustrate this invention. In these examples, the entire plant (flowers, stems, leaves, and roots) was used. The herbs were prepared by drying and milling.

### EXAMPLE 1

In this Example, each serving consisted of ¼ ounce water, 150 mg of Hoodia gordonii that had a peak at 1600 wave numbers of at least 0.05 absorbance units in an FTNIS, 200 mg of Cassia nomame, 2.337 mg glycerin, 0.007 mg menthol, and 67.5 mg natural cherry flavor. Sufficient citric acid was added to give a pH of 3.9. Inches measured were a sum of chest, arm, stomach, waist, hips, and thigh measurements.

In the first trial, 18 persons took 2 or 3 servings every day, immediately before eating a meal, for 1 to 4 weeks.

| | | | Initial | Weeks | |
|---|---|---|---|---|---|
| Participant | Sex | Age | Weight | of Use | Results |
| 1 | M | 11 | 168.6 | 3 | lost 2.8 lb., lost 3.5 in |
| 2 | M | 42 | 212.4 | 3 | gained 1.2 lb., lost 5.5 in |
| 3 | F | 41 | 185.6 | 3 | lost 0 lb., lost 5.75 in |
| 4 | F | 61 | 214.2 | 4 | lost 1.6 lb., lost 4.25 in |
| 5 | F | 36 | 134.2 | 3 | gained 1.6 lb., gained 1.6 in |
| 6 | F | 49 | 142.6 | 4 | lost 2.0 lb., lost 0.75 in. |
| 7 | F | 43 | 166.2 | 4 | gained 0.2 lb., lost 5.0 in |
| 8 | F | 35 | 186.8 | 4 | lost 5 lb., lost 2.75 in |
| 9 | F | 47 | 141.8 | 2 | lost 2.6 lb., lost 3.25 in |
| 10 | F | 56 | 196.2 | 2 | gained 1.6 lb., lost 5.75 in |
| 11 | F | 46 | 259.2 | 4 | lost 4.4 lb. lost 12.5 in. |
| 12 | M | ?? | 158.6 | 4 | lost 1.8 lb. lost 8.0 in. |
| 13 | M | 42 | 206.4 | 4 | lost 1.4 lb. lost 7.25 in |
| 14 | F | 28 | 155.2 | 2 | lost 0.8 lb. lost 1.25 in |
| 15 | M | 42 | 137 | 4 | lost 6.8 lb. lost 6.75 in |
| 16 | F | 40 | 206 | 3 | lost 0 lb., lost 0.75 in |
| 17 | F | 40 | 180.4 | 2 | gained 3.2 lb., lost 1.0 in |
| 18 | F | 42 | 264 | 4 | lost 0.2 lb., lost 2 in |

In the second trial, three women took a serving in liquid form immediately before meals twice a day for a week and two women took a capsule containing one serving immediately before meals twice a day for a week.

| | | | |
|---|---|---|---|
| Participant | Age | Initial Weight | Results |

Capsules:

| | | | |
|---|---|---|---|
| A | 52 | 171.6 | lost 2.2 lb. lost 3 in |
| B | 37 | 173.4 | Lost 0.2 lb. gained 4.5 in |

Liquid:

| | | | |
|---|---|---|---|
| A | 36 | 160 | lost 1.4 lb. lost 1.5 in |
| B | 30 | 230 | lost 4.4 lb. lost 0.5 in |
| C | 34 | 290 | lost 2.0 lb. lost 2 in |

Over 80% of participants reported a decrease in appetite in addition to the objective measurements.

This trail shows that the composition of this invention was more effective than the capsule in reducing the weight of the people taking it.

### EXAMPLE 2

In this study, 10 persons took liquid servings as described in Example 1 and 9 persons took gelatin capsules, each containing the same components in the same quantities as in one serving, but without the water. The liquid servings and the capsules were taken twice a day immediately before meals for six days. Each person was weighed each day. The 10 persons taking the liquid servings lost an average of 1.84 pounds each, while the 9 persons taking the capsules lost an average of 0.27 pounds each.

This study shows that the liquid servings were more effective than the capsules in reducing the weight of the people taking them. These results are especially significant because the average weight of the people taking the liquid servings was initially 173.32 while the average weight of the people taking the capsules was initially 203.28, and it is easier for heavier people to lose weight.

1. A composition comprising
(a) water;
(b) about 30 to about 1000 mg of Hoodia gordonii per ¼ ounce of water; and
(c) about 30 to about 1000 mg of Cassia nomame per ¼ ounce of water.

2. A composition according to claim 1 wherein the amount of said Hoodia.gordonii is about 100 to about 300 mg per ¼ ounce of water and the amount of said Cassia nomame is about 100 to about 300 mg per ¼ ounce of water.

3. A composition according to claim 1 wherein said Hoodia gordonii has a peak at 1600 wave numbers of at least 0.05 absorbance units in a Fourier Transform Near Infrared Spectrometer.

4. A composition according to claim 1 wherein said Hoodia Gordonii and Cassia nomame are prepared by drying and milling.

5. A composition according to claim 1 that contains an acid that lowers the pH of said composition to between about 3.0 and about 4.5.

6. A composition according to claim 1 wherein said composition includes about 23 to about 35 mg of citric acid per ¼ ounce.

7. A composition according to claim 1 that includes about 1800 to about 2600 mg per ¼ ounces of a flavor enhancer.

8. A composition according to claim 7 wherein said flavor enhancer is glycerin.

9. A composition according to claim 1 wherein said composition includes about 0.004 to about 0.12 mg of menthol per ¼ ounce.

10. A composition according to claim 1 wherein said flavoring agent is natural cherry.

11. A sealable container holding about 8 to about 16 ounces of a composition according to claim 1.

12. A composition according to claim 1 wherein the amount of said water is about 5 to about 15 cc.

13. A method of losing weight comprising drinking at least one serving of a composition according to claim 12 just before eating.

14. A method according to claim 13 wherein one serving of said composition is drunk twice per day if the person drinking it weighs up to 180 lbs and 2 two servings are drunk twice per day if the person drinking it weighs more than 180 lbs.

15. A composition comprising
(A) about 5 to about 15 cc of water;
(B) about 100 to about 300 mg of Hoodia gordonii;
(C) about 100 to about 300 mg of Cassia nomame;
(D) a sufficient amount of an acid to lower the pH below about 4.5;
(E) 0 to about 100 mg of a flavoring agent;
(F) 0 to about 2600 mg of a flavor enhancer; and
(G) 0 to about 0.12 mg of menthol.

16. A sealable container holding about 8 to about 16 ounces of a composition according to claim 15.

17. A method of losing weight comprising drinking at least one serving of a composition according to claim 15 twice per day just before eating.

18. A composition comprising
(A) about 5 to about 15 cc of water;
(B) about 100 to about 300 mg of Hoodia gordonii, where said Hoodia gordonii has a peak at 1600 wave numbers of at least 0.05 absorbance units in a Fourier Transform Near Infrared Spectrometer;
(C) about 100 to about 300 mg of Cassia nomame;
(D) an amount of citric acid sufficient to lower the pH to between about 3.0 and about 4.5;
(E) about 50 to about 100 mg of a flavoring agent; and
(F) about 1800 to about 2600 mg of glycerin; and
(G) 0.004 to about 0.12 mg of menthol.

19. A sealable container holding about 8 to about 16 ounces of a composition according to claim 18.

20. A method of losing weight comprising drinking at least two serving of a composition according to claim 18 twice per day just before eating.
